(19) United States
(12) Patent Application Publication
(10) Pub. No.: US 2010/0086519 A1
(43) Pub. Date: Apr. 8, 2010

(54) Treatment of Hepatitis C Infection With Metalloporphyrins

(75) Inventors: Herbert L. Bonkovsky, Charlotte, NC (US); Weihong Hou, Weddington, NC (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: The Charlotte-Mecklenburg Hospital Authority d/b/a Carolinas Medical Center

(21) Appl. No.: 12/572,651

(22) Filed: Oct. 2, 2009

Related U.S. Application Data
(60) Provisional application No. 61/102,503, filed on Oct. 3, 2008.

(51) Int. Cl. A61K 38/21; A61K 31/555; A61P 31/14

(52) U.S. Cl. 424/85.4; 514/185

(57) ABSTRACT

The present invention is directed to the treatment of hepatitis C infection with a metalloporphyrin. In particular, the present invention is based on the discovery that the NS5A protein plays a key role in HCV RNA replication by participating in polyprotein cleavage, interferon response and cellular signaling pathways. It has been found that metalloporphyrins, such as zinc porphyrins, induce post-translational down-regulation of HCV NS5A protein in an ubquitin-proteasome degradation pathway. That is, metalloporphyrins can be used to activate the ubiquitin-proteasomal pathway of NS5A protein catabolism. As a result, metalloporphyrins can be used to significantly suppress HCV viral replication in HCV infected cells.

### CROSS REFERENCE TO RELATED APPLICATIONS

This application is related to commonly owned copending Provisional Application Ser. No. 61/102,503, filed Oct. 3, 2008, incorporated herein by reference in its entirety, and claims the benefit of its earlier filing date under 35 U.S.C. 119(e).

### FEDERALLY SPONSORED RESEARCH OR DEVELOPMENT

This invention was made with United States Government support under RO1-DK38825 awarded by NIH/NIDDK. The United States Government has certain rights in the invention.

### FIELD OF THE INVENTION

The present invention relates to a method and formulation for the treatment of Hepatitis C infection.

### BACKGROUND OF THE INVENTION

Hepatitis C is a blood-borne infectious disease of the liver that is caused by the hepatitis C virus (HCV). HCV is a major cause of acute hepatitis and chronic liver disease, including cirrhosis and liver cancer. It is estimated that hepatitis C infects more than 180 million people worldwide and 4 million people in the United States. Hepatitis C is the leading cause of liver transplant in the United States with about 10,000 to 20,000 deaths a year in the United States being attributed to HCV infection.

HCV is a positive stranded RNA virus approximately 9.6 kb in length, and is the only known member of the hepacivirus genus in the family Flaviviridae. HCV encodes a single polyprotein of approximately 3010 amino acids that is then processed into structural (C, E1, E2) and nonstructural (NS2, NS3, NS4A, NS4B, NS5A and NS5B) proteins. The nonstructual viral proteins initiate the synthesis of negative strand RNA, which serves as a replication template for the generation of new positive strand viral genomes. The nonstructural 5A (NS5A) protein is a significant component of HCV proteins, and is a 447 amino acid phosphorylated zinc-metalloprotein with largely unknown functions. Recent studies have indicated that NS5A plays an important role in the replication of HCV, both directly, with regard to viral RNA replication, and indirectly, by modulating the host cell environment to favor the virus, and assembly of hepatitis C virus particles in JFH1-infected cells.

The most common form of treatment for HCV infection is a combination of pegylated interferon alpha and the antiviral drug ribavirin. Treatment periods generally run for a period of 24 or 48 weeks, depending on genotype. Indications for treatment include patients with proven hepatitis C virus infection and persistent abnormal liver function tests. However, this treatment fails to produce a sustained virological response in as many as 46% of treated persons. The treatment also has unpleasant side effects ranging from a ‘flu-like’ syndrome to severe adverse events including anemia, cardiovascular events and psychiatric problems such as suicide or suicidal ideation. The current treatments based on the combination of pegylated interferon alpha ribavirin are also expensive, and are generally too costly for patients in developing countries.

Thus, there still exists a need for new treatments for the treatments of HCV infection.

### BRIEF SUMMARY OF THE INVENTION

The present invention satisfies at least some of the aforementioned needs by providing a method and formulation for treating cells or a mammal suffering from HCV infection by reducing the level of NS5A cells in infected cells. The reduction of NS5A protein levels is achieved in accordance with the present invention by treating hepatitis C infection with a metalloporphyrin. In particular, the present invention is based on the discovery that the NS5A protein plays a key role in HCV RNA replication by participating in polyprotein cleavage, interferon response and cellular signaling pathways. It has been found that metalloporphyrins, such as zinc porphyrins, induce post-translational down-regulation of HCV NS5A protein in an ubquitin-proteasome degradation pathway. That is, metalloporphyrins can be used to activate the ubiquitin-proteasomal pathway of NS5A protein catabolism. As a result, metalloporphyrins can be used to significantly suppress HCV viral replication in HCV infected cells.

In the present invention, it has been found that metalloporphyrins reduce the stability of NS5A protein by decreasing the protein's half life from about 19.8 hours to about 1.2 hours, and significantly induces polyubquitination of NS5A. As a result, HCV RNA replication can be significantly reduced. Ubquitin (Ub) was first identified as a highly-conserved small protein in eukaryotic cells that is composed of 76 amino acids with a predicted molecular weight of 8.5 kD. The ubquitin-proteasome degradation pathway has been well accepted as an important regulatory system in many cellular processes such as cell cycle, DNA repair, embryogenesis, the regulation of transcription and apoptosis. In the ubiquitin-proteasome pathway, protein substrates are first marked for degradation by covalent linkage to multiple molecules of ubiquitin (polyubiquitination) and then are hydrolyzed by the 26 S proteasome, a 2000 kDa ATP-dependent proteolytic complex. Accordingly, inducing polyubquitination of the NS5A protein can lead to a reduction in HCV RNA replication. It has further been found that metalloporphyrins can be used to down-regulate NS5A protein levels in a dose-dependent fashion in human hepatoma cells stably expressing HCV proteins.

In a preferred embodiment, the method of treating HCV infection comprises treating infected cells with a zinc porphyrin, such as zinc mesoporphyrin (ZnMP) or zinc protoporphyrin. It has been found that both ZnMP and ZnPP induce polyubquitination of NS5A and display anti-viral activity. Zinc porphyrins have been found to be particularly useful in the treatment of HCV infection because they are generally readily taken up by intact liver cells.

In one embodiment, the present intention is also directed to formulations for the treatment of HCV infection. In one particular embodiment, the present invention provides a formulation comprising a zinc porphyrin and albumin. Zinc porphyrin, when administered as an albumin complex, is nontoxic, and is taken up preferentially by the liver and spleen.

In a further embodiment, it has been discovered that metalloporphyrins can be used in combination with other antiviral remedies to provide an additive or synergistic effect. For instance, formulations in accordance with present invention can include a combination of a metalloporphyrin with one or more interferons such as α-interferon, β-interferon and/or γ-interferon.

Accordingly, the present invention provides methods and formulations for the treatment of HCV infection.

### BRIEF DESCRIPTION OF THE SEVERAL VIEWS OF THE DRAWING(S)

Having thus described the invention in general terms, reference will now be made to the accompanying drawings, which are not necessarily drawn to scale, and wherein:

FIG. 1A depicts a Western blot and accompanying bar graph that shows the effects of various concentrations of zinc mesoporphyrin on NS5A in 9-13 cells;

FIG. 1B depicts a Western blot and accompanying bar graph that shows the effects of various concentrations of zinc mesoporphyrin on NS5A in Con1 cells;

FIG. 1C depicts a Western blot and accompanying bar graph that shows the effects of various concentrations of zinc mesoporphyrin on core protein levels in CNS3 cells;

FIG. 1D depicts a Western blot and accompanying bar graph that shows the effects of various concentrations of zinc mesoporphyrin on NS5A and core protein levels in Huh-7/T7 cells transfected with pFK-Con1/GND.

FIG. 2A depicts a Western blot and accompanying bar graph that shows the effects of zinc mesoporphyrin on NS5A protein levels in comparison to DMSO, mesoporphyrin, and zinc chloride;

FIG. 2B depicts a Western blot and accompanying bar graph that shows the effects of zinc mesoporphyrin on NS5A protein levels in comparison to tin mesoporphyrin;

FIG. 3 depicts a Western blot and accompanying bar graph that shows the effects of zinc chelator, N, N, N, N-tetrakis-(2-pyridylmethyl)ethylenediamine (TEPN) on NS5A protein levels;

FIG. 4A depicts a Western blot of the effects of zinc mesoporphyrin on the protein levels of NS5A in the presence of cycloheximide [CHX], an inhibitor of protein synthesis, to determine the approximate half life of NS5A protein in zinc mesoporphyrin-treated hepatocytes;

FIG. 4B depicts a bar graph that shows the intensities of bands in panel A;

FIG. 4C is a graph illustrating the effect of zinc mesoporphyrin on the half life of NS5A protein;

FIG. 5A is a Western blot that shows the effects on NS5A protein levels in 9-13 cells that were treated with ZnMP and different concentrations of epoxomicin or MG132;

FIG. 5B is a normalized bar graph showing the effects on NS5A protein levels in 9-13 cells that were treated with ZnMP and different concentrations of epoxomicin or MG132;

FIG. 5C is a Western blot that shows the effects on NS5A protein levels in 9-13 cells that were treated with epoxomicin or MG132 in the absence of ZnMP;

FIG. 6A illustrates a Western blot analysis depicting degradation of NS5A following ZnMP treatment before immunoprecipitation (IP), showing down-regulation of NS5A by ZnMP;

FIG. 6B illustrates a immunoprecipitation and an immunoblotting (IB) analysis depicting polyubiquitination of NS5A following ZnMP treatment in comparison to a control;

FIG. 6C illustrates an immunoblotting (IB) analysis depicting that NS5A proteins were immunoprecipitated in panel B.

FIG. 7A is a bar graph depicting the dose effect of ZnMP on HCV RNA in Con1 cells.

FIG. 7B depicts a Western blot and accompanying bar graph that shows the effects of zinc mesoporphyrin on HCV protein levels in Con1 cells;

FIG. 7C depicts a Western blot and accompanying bar graph that shows the effects of various concentrations of zinc mesoporphyrin on NS5A and core protein levels in Huh-7/T7 cells transfected with pFK-Con1/GND.

FIG. 7D depicts a Western blot and accompanying bar graph that shows the effects of various concentrations of zinc mesoporphyrin on NS5A and core protein levels in Huh-7/T7 cells transfected with pFK-Con1/GDD.

FIG. 8A depicts a Western blot analysis that shows the effects of ZnMP on NS5A and core protein levels after 4 h treatment in the JFH1-based cell culture system;

FIG. 8B depicts a bar graph that shows the effects of ZnMP on NS5A and core protein levels after 4 h treatment in the JFH1-based cell culture system;

FIG. 8C depicts a Western blot analysis that shows the effects of ZnMP on NS5A and core protein levels after 24 h treatment in the JFH1-based cell culture system;

FIG. 8D depicts a bar graph that shows the effects of ZnMP on NS5A and core protein levels after 24 h treatment in the JFH1-based cell culture system;

FIG. 8E is a bar graph depicting the effect of ZnMP on HCV RNA replication in Huh-7.5 cells transfected with J6/JFH1 RNA.

FIG. 8F is a bar graph depicting the effect of ZnMP on HCV RNA replication in Huh-7.5 cells infected with J6/JFH1 HCV.

FIG. 9A depicts a Western blot and accompanying bar graph that shows the effects of alpha interferon on NS5A protein levels in 9-13 cells;

FIG. 9B depicts a Western blot and accompanying bar graph that shows the effects of zinc mesoporphyrin on NS5A protein levels in 9-13 cells;

FIG. 9C depicts a Western blot and accompanying bar graph that shows the combined effects of alpha interferon and zinc mesoporphyrin on NS5A protein levels in 9-13 cells; and

FIG. 10 is a bar graph depicting the dose and time course effects of ZnMP on cytotoxicity.

### DETAILED DESCRIPTION OF THE INVENTION

The present invention now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

The present invention is directed to a method of treating hepatitis C viral infection with a metalloporphyrin. In one embodiment, the present invention is directed to a method of treating cells that are infected with HCV, and in particular to a method of treating hepatitis C viral infection in a patient with a metalloporphyrin. Embodiments of the present invention are also directed to pharmaceutical formulations comprising a metalloporphyrin for the treatment of hepatitis C viral infection. In a preferred embodiment, the present invention is directed to a method of treating hepatitis C viral infection with a zinc porphyrin.

The inventors of the present invention have discovered that metalloporphyrins can be used to treat HCV infection by inhibiting HCV replication. NS5A plays a critical role in the replication of HCV by participating in polyprotein cleavage, interferon response, and cellular signaling pathways. In the present invention, it is believed that HCV replication is reduced by controlling the amount of HCV NS5A protein in the host cell with a metalloporphyrin. While not wishing to be bound by theory, it is believed that metalloporphyrins suppress HCV viral replication in HCV infected cells by mediating ubiquitin-proteasome degradation pathway of NS5A proteins. As a result, the amounts of NS5A proteins in infected cells can be significantly reduced thereby reducing HCV RNA replication.

In the present invention, it has been found that metalloporphyrins reduce the stability of NS5A protein by decreasing the protein's half life from about 19.8 hours to about 1.2 hours, and significantly induces polyubquitination of NS5A. As a result, HCV RNA replication can be significantly reduced. Ubquitin (Ub) was first identified as a highly-conserved small protein in eukaryotic cells that is composed of 76 amino acids with a predicted molecular weight of 8.5 kD. The ubquitin-proteasome degradation pathway has been well accepted as an important regulatory system in many cellular processes such as cell cycle, DNA repair, embryogenesis, the regulation of transcription and apoptosis. In the ubiquitin-proteasome pathway, protein substrates are first marked for degradation by covalent linkage to multiple molecules of ubiquitin (polyubiquitination) and then are hydrolyzed by the 26 S proteasome, a 2000 kDa ATP-dependent proteolytic complex. Accordingly, inducing polyubquitination of the NS5A protein can lead to a reduction in HCV RNA replication. It has further been found that zinc porphyrins can be used to down-regulate NS5A protein levels in a dose-dependent fashion in human hepatoma cells stably expressing HCV proteins.

In one embodiment, the half-life of NS5A protein is reduced to between about 0.5 to 3 hours, and preferably is reduced to about 0.8 to 1.5 hours, and more preferably from about 1 to 1.2 hours. As noted above, reduction of the half-life of the NS5A protein has a significant affect on the ability of HCV to replicate.

Metalloporphyrins are macrocycle compounds with bridges of one carbon atom or one nitrogen atom respectively, joining the pyrroles to form the characteristic tetrapyrrole ring structure in which a metal ion is inserted into the tetrapyrrole ring. The porphyrin structure may also include various ligands and moieties that are attached thereto. Examples of suitable metals may include, but are not limited to, Fe, Co, Zn, Mn, Cr, Ni, Mg, and Cu. In a preferred embodiment, metallophorphyrins for use in the present invention are selected from the group consisting of zinc mesoporphyrins, zinc protoporphyrins, heme and cobalt protoporhyrins, and combinations thereof. Other organo metallic derivates of metalloporphyrins that may be used in the present invention include, for example, zinc deuteroporphyrin, zinc deuteroporphyrin bisglycol, cobalt protoporphyrin, cobalt mesoporphyrin, cobalt deuteroporphyrin, cobalt deuteroporphyrin bisglycol, heme, iron mesoporphyrin, iron deuteroporphyrin, and iron deuteroporphyrin bisglycol.

The present invention provides a method of treating and/or ameliorating HCV infection by administering a therapeutically effective amount and/or a prophylactic amount of a formulation containing a metalloporphyrin or a pharmaceutically acceptable salt thereof, to a sufferer in need thereof. By “therapeutically effective amount” it is meant an amount of the active ingredient (e.g., metalloporphyrin or a pharmaceutically acceptable salt thereof) to a mammal is effective to treat and/or ameliorate HCV infection. In a preferred embodiment, the present invention is directed to a method of treating and/or ameliorating HCV infection in a human patient.

In one embodiment, dosage forms (compositions) of the metalloporphyrin formulation of the present invention may contain about 0.1 to 20 mg/kg body weight/day of active ingredient per unit, and in particular, from about 10 to 80 milligrams of active ingredient per unit, such as from about 14 to 75 milligrams, 20 to 70 milligrams, 35 to 65 milligrams, 40 to 50 milligrams, or from about 40 to 45 milligrams of active ingredient per unit. In one embodiment, a unit dose of metalloporphyrin will generally contain from 5 to 1000 mg and preferably will contain from 30 to 500 mg, in particular 50, 100, 150, 200, 250, 300, 350, 400, 450, or 500 mg.

In one embodiment, the formulation containing the metalloporphyrin may be administered once or more times a day for example 2, 3 or 4 times daily, and the total daily dose for a 70 kg adult will normally be in the range 100 to 3000 mg. Alternatively the unit dose may contain from 2 to 20 mg of a metalloporphyrin and be administered in multiples, if desired, to give the preceding daily dose. In these pharmaceutical compositions, the active ingredient will ordinarily be present in an amount of about 0.5-95% by weight based on the total weight of the formulation.

According to various embodiments, formulations of the present invention can be administered to a patient in need thereof in a variety of manners, including enterally and intravenously. For instance, a formulation according to the present invention can be prepared in the form of a liquid, solid, gel, or a combination thereof. In a preferred embodiment, formulations in accordance with the present invention are provided in a solid dose form, such as a tablet or capsule.

For use in the treatment of HCV infection, by way of general guidance, a daily oral dosage of the metalloporphyrin can generally range from about 0.1 to 1000 mg/kg of body weight.

For instance, for oral administration in the form of a tablet or capsule, the active ingredient can be combined with an oral, non-toxic, pharmaceutically acceptable, inert carrier, including but not limited to, lactose, starch, sucrose, glucose, methyl cellulose, magnesium stearate, dicalcium phosphate, calcium sulfate, mannitol, sorbitol and the like. Additionally, when desired or necessary, suitable binders, lubricants, disintegrating agents, and coloring agents can also be incorporated into the mixture. Suitable binders may include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms may include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

In one embodiment, the metalloporphyrin is administered in a formulation in which it is bound to human serum albumin (HAS) at about 10:1 to 1:1 molar ratios. The use of human serum albumin helps to enhance the uptake of the metalloporphyrin into liver cells. Additionally, when administer as an albumin complex, the formulation is nontoxic, and is taken up preferentially by the liver and spleen.

In some embodiments, the formulations containing metalloporphyrins of the present invention may also be coupled with soluble polymers as targetable drug carriers. Such polymers can include, for example, polyvinylpyrrolidone, pyran copolymer, polyhydroxypropylmethacrylamide-phenol, polyhydroxyethylaspartamidephenol, or polyethyleneoxide-polylysine substituted with palmitoyl residues. In one embodiment, formulations of the present invention may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

In certain embodiments of the present invention, the metalloporphyrns of the present invention and/or compositions thereof can be used in combination therapy with at least one other therapeutic agent. A compound of the invention and/or composition thereof and the therapeutic agent can act additively or, more preferably, synergistically. The compound of the invention and/or a composition thereof may be administered concurrently with the administration of the other therapeutic agent(s), or it may be administered prior to or subsequent to administration of the other therapeutic agent(s).

In one embodiment, the compounds of the invention and/or compositions thereof are used in combination therapy with other antiviral agents or other therapies known to be effective in the treatment or prevention of HCV. As a specific example, the present invention provides a method of treating HCV infection by administering a combination of a metalloporphyrin compounds of the invention and/or compositions thereof may be used in combination with known antivirals, such as interferon-α, ribavirin (see, e.g., U.S. Pat. No. 4,530,901), Telaprevir, HCV Protease, and polymerase inhibitors.

In yet as another specific example, the compounds of the invention and/or compositions thereof may be used in combination with interferons such as α-interferon, β-interferon and/or γ-interferon. The interferons may be unmodified, or may be modified with moieties such as polyethylene glycol (pegylated interferons). Many suitable unpegylated and pegylated interferons are available commercially, and include, by way of example and not limitation, recombinant interferon alpha-2b such as Intron-A interferon available from Schering Corporation, Kenilworth, N.J., recombinant interferon alpha-2a such as Roferon interferon available from Hoffmann-La Roche, Nutley, N.J., recombinant interferon alpha-2C such as Berofor alpha 2 interferon available from Boehringer Ingelheim Pharmaceutical, Inc., Ridgefield, Conn., interferon alpha-n1, a purified blend of natural alpha interferons such as Sumiferon available from Sumitomo, Japan or as Wellferon interferon alpha-n1 (INS) available from the Glaxo-Wellcome Ltd., London, Great Britain, or a consensus alpha interferon such as those described in U.S. Pat. Nos. 4,897,471 and 4,695,623 (especially Examples 7, 8 or 9 thereof) and the specific product available from Three Rivers Pharmaceuticals, Cranberry Township, Pa., or interferon alpha-n3 a mixture of natural alpha interferons made by Interferon Sciences and available from the Purdue Frederick Co., Norwalk, Conn., under the Alferon Trade name, pegylated interferon-2b available from Schering Corporation, Kenilworth, N.J. under the tradenanie PEG-Intron A and pegylated interferon-2a available from Hoffmann-LaRoche, Nutley, N.J. under the trade name Pegasys.

In one particular embodiment, the present invention provides a pharmaceutical formulation for the treatment of HCV infection comprising a combination of a metalloporphyrin and an interferon. In one embodiment, the formulation comprises from about 0.1 to 20 mg/kg BW of metalloporphyrin per unit, and about 0.5 to 5 mcg/kg BW of interferon per unit.

The following examples are provided for the purpose of illustration only and should not be construed as limiting the invention in any way.

### EXAMPLES

The following is a brief description of the reagent and procedures used to evaluate the use of metalloporphyrins in the treatment of HCV infection.

Reagents and Antibodies

Zinc mesoporphyrin (ZnMP) was purchased from Frontier Scientific (Logan, Utah).

Zinc protoporphyrin (ZnPP) was purchased from Frontier Scientific (Logan, Utah).

Tin mesoporphyrin (SnMP) mesoporphyrin was purchased from Frontier Scientific (Logan, Utah).

Dimethyl sulfoxide (DMSO) was purchased from Fisher Biotech (Fair Lawn, N.J.).

Mouse anti-HCV NS5A monoclonal antibody was from Virogen (Watertown, Mass.).

Rabbit anti-HCV NS5A polyclonal antibody was from Virogen (Watertown, Mass.).

Mouse anti-HCV core monoclonal antibody was from Affinity BioReagent (Golden, Colo.

Goat anti-human GAPDH polyclonal antibody was purchased from Santa Cruz (Santa Cruz, Calif.).

ECL-Plus was from Amersham (Piscataway, N.J.).

Epoxomicin and MG132 were from Sigma-Aldrich (St. Louis, Mo.).

BCA protein assay reagent was from Pierce (Rockford, Ill.).

Dulbecco's modified Eagle's medium (DMEM) and fetal bovine serum (FBS) were from HyClone (Logan, Utah).

TRIzol and zeocin were purchased from Invitrogen (Carlsbad, Calif.).

G418 was from Gibco (Grand Island, N.Y.).

CellTiter-Glo® Reagent was from Promega (Madison, Wis.).

Primers were synthesized by Integrated DNA Technologies (Coralville, Iowa).

4-15% gradient SDS-PAGE gels and ImmunBlot PVDF membranes were from purchased from Bio-Rad (Hercules, Calif.).

Cell Culture

Cell lines 9-13, CNS3 and Huh-7/T7 were provided by Dr. Ralf. Bartenschlager (University of Heidelberg, Heidelberg, Germany). 9-13 cells, containing a replicating HCV nonstructural region, stably express HCV NS3 to NS5B. CNS3 cells stably express HCV core to NS3 (amino acid residues 1 through 1233 of the Con1 isolate; Gene Bank accession number AJ238799). Huh-7/T7 cells constitutively express the bacteriophage T7 RNA polymerase. The cells were maintained in DMEM supplemented with 10% (v/v) FBS and 500 μg/mL G418 for 9-13 cells, 10 μg/mL zeocin for CNS3 cells or 5 μg/mL zeocin for Huh-7/T7 cells. The Con 1 (subtype 1b) full length replicon Huh-7.5 cells (Con1 cells) were provided by Dr. Charles M. Rice (The Rockefeller University, New York, N.Y.). The Con1 cell line is a Huh-7.5 cell population containing the full-length HCV genotype 1b replicon with the highly adaptive serine to isoleucine substitution at amino acid 2204 of the polypeptide. The Con1 cells were maintained in DMEM supplemented with 10% (v/v) FBS and 0.1 mM nonessential amino acids, 100 units/mL penicillin, 100 μg/mL streptomycin, and selection antibiotic 750 μg/mL G418.

Western Blots

Western blots were performed using the standard protocols of our laboratory as described in Hou et al. Zinc mesoporphyrin induces rapid and marked degradation of the transcription factor Bach1 and up-regulates HO-1. Biochim Biophys Acta 2008; 1779:195-203. In brief, total proteins (30-50 μg) were separated on 4-15% gradient SDS-PAGE gels. After electrophoretic transfer onto ImmunBlot PVDF membrane, membranes were blocked for 1 hour in PBS containing 5% nonfat dry milk and 0.1% Tween-20, and then incubated overnight with primary antibody at 4° C. The dilutions of the primary antibodies were as follows: 1:2000 for anti-HCV NS5A and anti-GAPDH antibodies, and 1:5000 for anti-HCV core antibody. The membranes were then incubated for 1 hour with horseradish peroxidase-conjugated secondary antibodies (dilution 1:10,000). Finally, the bound antibodies were visualized with the ECL-Plus chemiluminescence system according to the manufacturer's protocol. A Kodak 1DV3.6 computer-based imaging system (Rochester, N.Y.) was used to measure the relative optical density of each specific band obtained after Western blotting. Data are expressed as percentages of the controls (corresponding to the value obtained with the vehicle-treated cells), which were assigned values of one.

Transfection of pFK-Con1/GDD or pFK-Con1/GND

The pFK-Con1/GDD and pFK-Con1/GND constructs (genotype 1b) were gifts of Dr. R. Bartenschlager (University of Heidelberg, Heidelberg, Germany). pFK-Con1/GND construct was a replication-deficient variant of pFK-Con1/GDD with a single amino substitution, which changed the GDD motif of the NS5B polymerase active site to GND. Transfection of pFK-Con1/GDD or pFK-Con1/GND was performed as described in the following procedure. In brief, Huh-7/T7 cells, stably expressing the T7 RNA polymerase, were seeded in 24-well plates one day before transfection, and grown up to 80% confluence. Cells were transfected with 0.8 ug/well of pFK-Con1/GDD or pFK-Con1/GND by Lipofectamine and Plus Reagent (Invitrogen, Carlsbad, Calif.) for 48 h according to the manufacturer's instructions.

In Vitro Transcription, HCV RNA Transfection and Infection

The HCV infectious clone pJ6/JFH1 was provided by Dr. C. Rice (the Rockefeller University, New York, N.Y.). The full-length chimeric genome was constructed with the use of the core-NS2 gene regions from the infectious J6 (genotype 2a) and NS3-NS5B gene regions from the infectious JFH1 (genotype 2a) as described by Lindenbach et al. To generate HCV J6/JFH1 RNA, the pJ6/JFH1 plasmid was linearized with XbaI, and purified by ethanol precipitation, digestion with proteinase K, and phenol-chloroform extraction. The linearized plasmid was used as a template for in vitro transcription using the MEGAscript T7 kit (Ambion, Austin, Tex.). For HCV RNA transfection, Huh-7.5 cells were plated in 24-well plates one day prior to transfection and transfected at 70˜80% confluence. Cells were transfected at an RNA/lipofectamine ratio of 1:2 by using 2 μg/well of HCV RNA and 4 uL/well Lipofectamine 2000 (Invitrogen, Carlsbad, Calif.) for 48 h. For HCV infection, cell culture supernatants from the cells transfected with HCV RNA for 48 h were collected and filtered through a 0.20 μm filter, and infected naïve Huh-7.5 cells in 24-well plates for 72 h.

Immunoprecipitation (IP)

Immunoprecipitation was carried out according to the Manufacturer's protocol. Briefly, cells were harvested in cold radioimmunoprecipitation assay (RIPA) buffer (50 mM Tris-HC1 [pH 7.4], 150 mM NaCl, 1 mM EDTA, 0.25% sodium deoxycholate, 1% IGEPAL CA-630, 1 mM PMSF, 1 mM NaF, 1 mM Na₃VO₄ and 1 μg/ml each of aprotinin, leupeptin, and pepstatin). The samples were pre-cleared with Protein A/G Agarose for 10 min at 4° C. and subsequently incubated while gently rotating at 4° C. overnight with primary antibody, followed by Protein A/G beads for 1 h at 4° C. Beads were spun down and washed twice with RIPA buffer. Protein samples were separated by electrophoresis on 4-15% SDS-PAGE gels, and transferred to PVDF membrane, conjugated ubiquitin was detected as described for Western blot analysis using anti-ubiquitin antibody.

Quantitative RT-PCR

Total RNA from treated cells was extracted and cDNA was synthesized as described in Hou et al. Real time quantitative RT-PCR was performed using a MyiQ™ Single Color Real-Time PCR Detection System from Bio-Rad (Hercules, Calif.) and iQ™ SYBR Green Supermix Real-Time PCR kit (Bio-Rad). Samples without template and without reverse transcriptase were included as negative controls, which, as expected, produced negligible signals (Ct values>35). Fold-change values were calculated by comparative Ct analysis after normalizing for the quantity of GAPDH in the same samples.

Protein Half-Life Determination

9-13 cells were incubated with 100 ug/mL cycloheximide (CHX) in the presence or absence of 10 uM ZnMP. Western blots were performed using anti-HCV NS5A and anti-GAPDH antibodies. Band intensities of Western blots were measured by densitometric analysis. GAPDH bands were used as internal controls to correct for protein loadings.

Cell Viability Assay

The effect of ZnMP on cytotoxicity of treated cells was measured using CellTiter-Glo® Reagent by determining the number of viable cells based on quantitation of the ATP present, which signaled the presence of metabolically active cells. Cells were plated into a 96-well plate with 2500 to 5000 cells/well 24 hours before treatment. Cells were treated with indicated concentrations of ZnMP for 2, 6 and 24 hours in triplicate, an equal volume of CellTiter-Glo® Reagent was added to each well of cell culture medium. The luminescence was read on a Synergy HT microplate reader from Bio-Tek (Winooski, Vt.) with integration time set for 0.25 to 1 second. Decreases in luminescence were taken as an index of cellular cytotoxicity.

Statistical Analysis

Initial analysis showed that results were normally distributed. Therefore, parametric statistical procedures were used. The Student's t-test or ANOVA was used (as appropriate) to analyze the differences between samples. Values of P<0.05 were considered statistically significant. Experiments were repeated at least three times with similar results. All experiments included at least triplicate samples for each treatment group. Representative results from single experiments are presented. Statistical analyses were performed with JMP 4.0.4 software from SAS Institute (Cary, N.C.).

### Example 1

In this example, the down-regulation of HCV proteins by ZnMP was investigated. NS5A protein levels in 9-13 and Con1 cells, core protein levels in CNS3 cells, and NS5A and core protein levels in Huh-7/T7 cells transfected with pFK-Con1/GND (a plasmid encoding a replication deficient variant of Con1) exposed to different concentrations of ZnMP (0, 1, 5, 10 μM) for 4 hours were evaluated. After treatment, the cells were harvested and the total protein was isolated. Proteins were separated on 4-15% SDS-polyacrylamide gel, transferred to a PVDF membrane, and probed with anti-HCV NS5A, anti-HCV core or GAPDH specific antibodies, bands corresponding to NS5A, core or GAPDH were detected by the ECL-Plus chemiluminescence as described above. In the Figs., the amounts of NS5A or core protein levels were normalized to GAPDH which did not vary with treatment. Values for cells treated with vehicle only were set equal to 1. Data are presented as means±SE from triplicate samples. * differs from vehicle only, P<0.05.

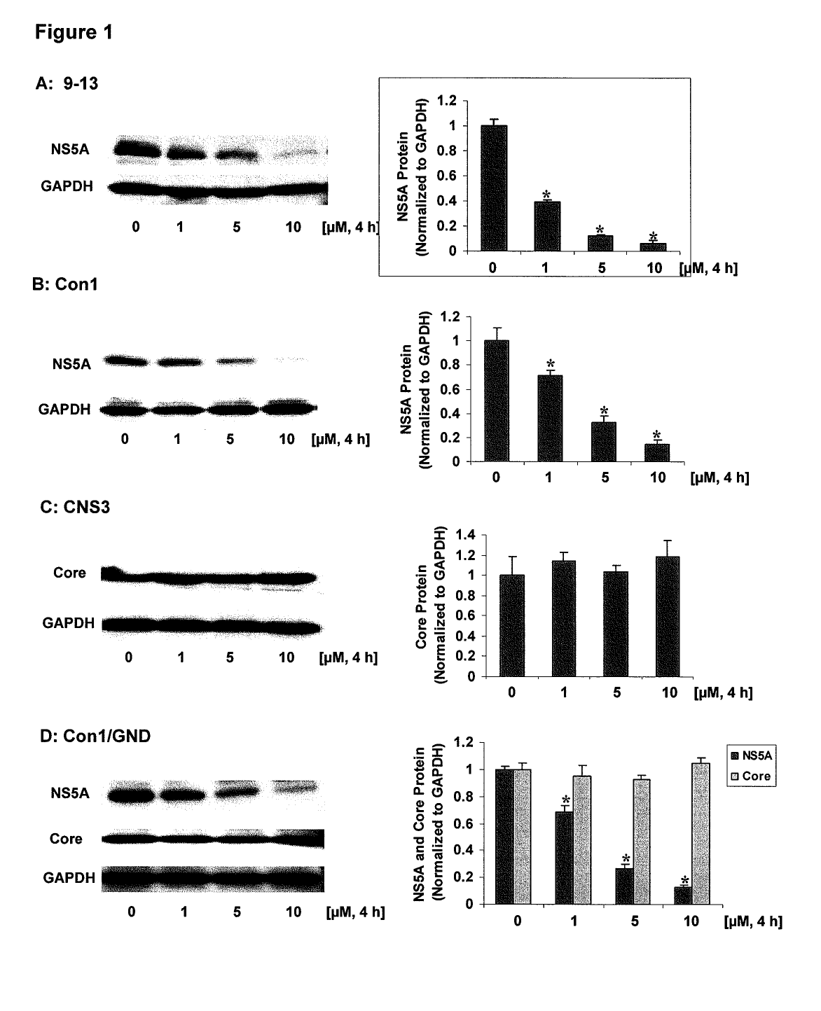

As shown in the western blots of FIGS. 1A and 1B, cells exposed to ZnMP led to a marked decrease of NS5A protein levels in 9-13 and Con1 cells in a dose-dependent fashion. Further, the effect of the zinc porphyrin was selective and specific: there were no detectable effects of ZnMP on HCV core protein levels in CNS3 cells and Huh-7/T7 cells transfected with pFK-Con1/GND as can be seen in FIGS. 1C and 1D. The accompanying bar graphs show that the administration of metalloporphyrins can significantly reduce the levels of NS5A proteins. For example, NS5A protein levels were reduce from about 60% for a dosage of about 1 μM of the porphyrin to about 80 to 90% for a dosage of about 5 μM of the porphyrin. In particular, it can be seen that the administration of a dose of 10 μM of the zinc porphyrin reduced the level of NS5A proteins from about 90 to 95% after 4 hours.

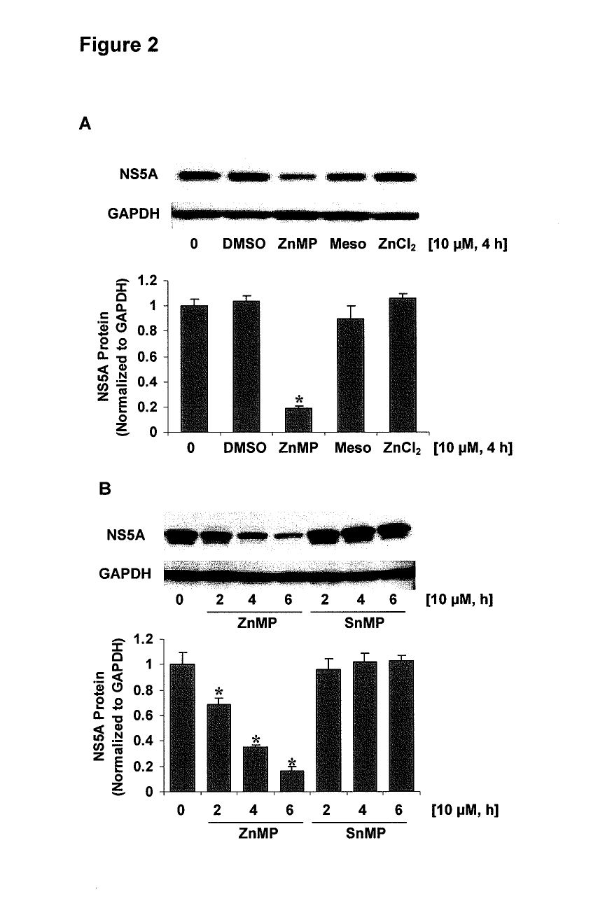

In addition, it was observed that protein levels were not affected by 10 μM free mesoporphyrin or ZnCl₂. In FIG. 2A, the effects of zinc porphyrin on NS5A protein levels in comparison to DMSO, mesoporphyrin, and zinc chloride are compared. In this Example, 9-13 cells were exposed to ZnMP, free mesoporphyrin (Meso) or with ZnCl₂ for 4 hours, followed by extraction of total proteins. Western blot was performed using anti-HCV NS5A and GAPDH specific antibodies. It can be seen that while ZnMP decreased the levels of NS5A protein, meso porphyrin and zinc alone had relatively little if any effect on NS5A protein levels.

Tin mesoporphyrin (SnMP), another competitive HO inhibitor, has been reported recently to down-regulate Bach1 protein levels and induce the HO-1 gene expression in NIH 3T3 cells. In FIG. 2B the effects ZnMP and SnMP in the down-regulation of NS5A in 9-13 cells was compared. 9-13 cells were treated with 10 μM ZnMP or with 10 μM SnMP for indicated times (0, 2, 4, 6 h), and then the cells were harvested using harvest buffer containing the protease inhibitor cocktail. 50 μg of total proteins were loaded on a 4-15% SDS-polyacrylamide gel, transferred to a PVDF membrane, and detected with anti-NS5A, and anti-GAPDH specific antibodies, and then developed with ECL Plus chemiluminescence. As shown in FIG. 2B, ZnMP markedly and rapidly decreased NS5A protein levels after exposure to ZnMP for as little as 2 hours. In contrast, no detectable effects of SnMP on NS5A protein levels were observed. The relative amounts of NS5A protein were normalized to those for GAPDH, which did not vary with treatment. Values for cells treated with vehicle only were set equal to 1. Data are presented as means±SE from triplicate samples. * differs from vehicle only, P<0.05.

### Example 2

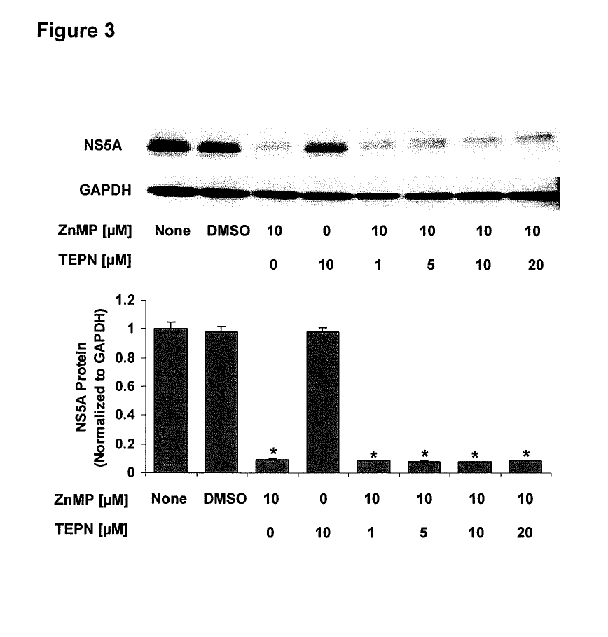

In Example 2, effect of zinc chelator, N, N, N, N-tetrakis-(2-pyridylmethyl) ethylenediamine (TEPN) on NS5A protein levels was investigated. 9-13 cells were treated with indicated concentrations of TEPN 30 min before ZnMP treatment, the cells were subsequently exposed to ZnMP or to vehicle (DMSO) alone as control for 4 h. Total proteins were extracted. NS5A and GAPDH protein levels were measured by Western blot. The bar graphs show quantitative results. The relative amounts of NS5A protein were normalized to those for GAPDH, which did not vary with treatment. The band intensity of NS5A from vehicle alone was set equal to 1. * differs from vehicle only, P<0.05. As shown in FIG. 3, zinc chelator TEPN did not affect ZnMP-mediated profound down-regulation of NS5A protein levels in 9-13 cells.

### Example 3

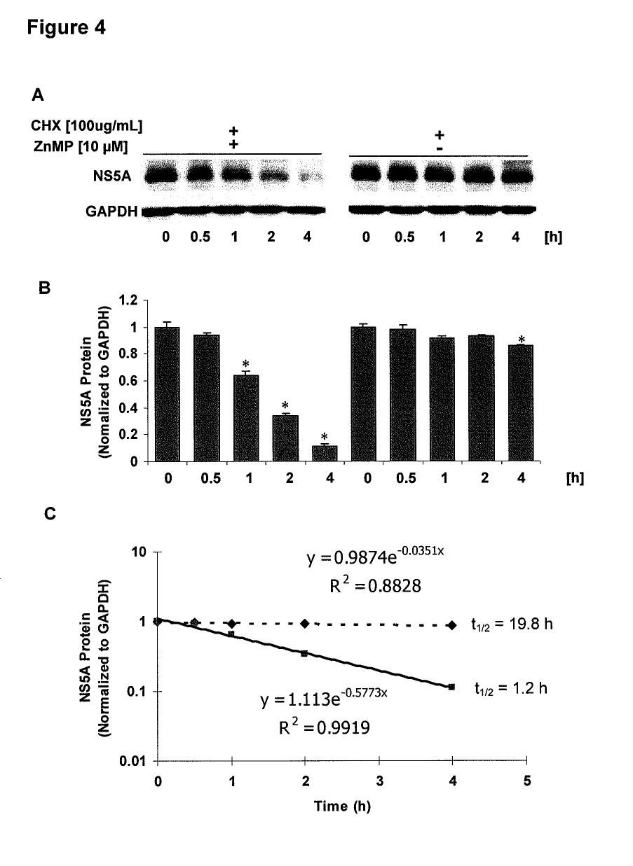

In Example 3, the down-regulation of NS5A protein by ZnMP was investigated to determine whether the down-regulation occurs at a post-translational level. 9-13 cells were treated with 100 ug/mL cycloheximide (CHX) and with or without 10 μM ZnMP for the indicated periods (0, 0.5, 1, 2, 4 h), and then cells were harvested and total proteins were isolated. Proteins were separated on 4-15% SDS-polyacrylamide gel, transferred to a PVDF membrane, anti-HCV NS5A or GAPDH specific antibodies were used to detect NS5A or GAPDH protein levels by Western blot. As shown in FIGS. 4A and 4B, NS5A protein levels in 9-13 cells treated with ZnMP and cycloheximide (CHX) were greatly and rapidly reduced. NS5A protein levels in 9-13 cells that were not treated with ZnMP were also decreased by CHX, but to a much less extent. ZnMP at a concentration of 10 μM decreased the NS5A protein half life (t_{1/2}) from 19.8 hours to 1.2 hours (FIG. 4C). In FIGS. 4B and 4C, the intensities of bands in FIG. 4A were quantified by densitometry. The band intensity of NS5A from untreated sample (0 h) was set at 1.

### Example 4

Two distinct systems for protein degradation have been found in mammals: the lysosome system and the ubiquitin-proteasome system. Proteasome-dependent degradation pathway is one of the major proteolytic pathways. To understand whether degradation of NS5A protein by ZnMP is proteasome dependent, 9-13 cells were treated with ZnMP (5, 10 μM) and selected proteasome inhibitors, epoxomicin (5, 10 μM) and MG132 (10, 20 μM). 9-13 cells were treated with indicated concentrations of MG132 or epoxomicin 30 min before ZnMP treatment. The cells were subsequently exposed to ZnMP or with vehicle alone as control for 4 hours. Total proteins were extracted. NS5A and GAPDH protein levels were measured by Western blot as described in above.

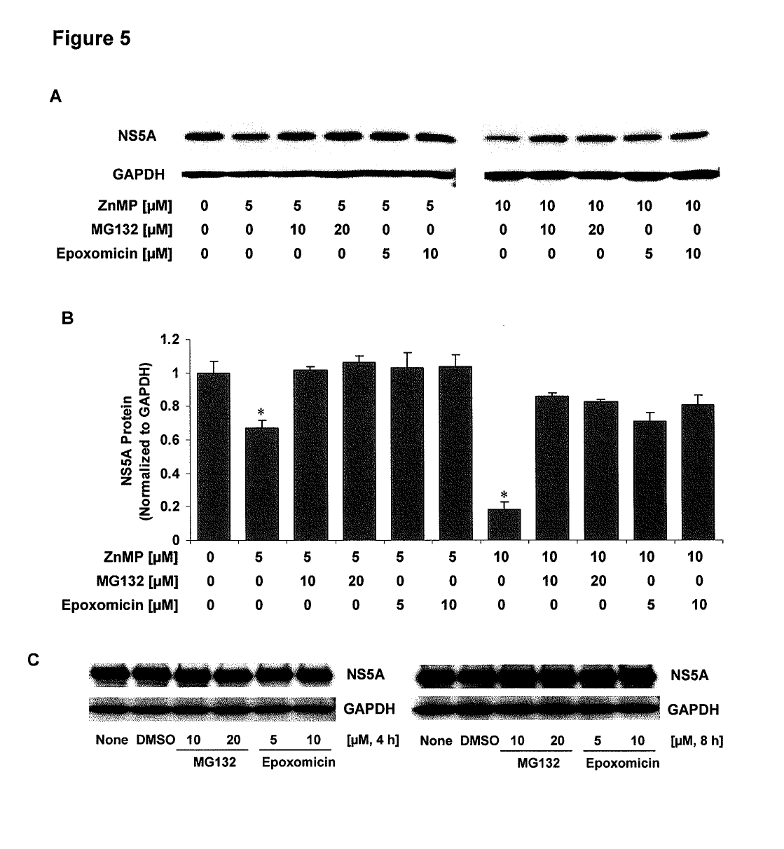

FIG. 5A is a Western blot that shows the effects on NS5A protein levels in 9-13 cells that were treated with ZnMP (5, 10 μM) and different concentrations of epoxomicin (5, 10 μM) or MG132 (10, 20 μM). FIG. 5B is a normalized bar graph of the results depicted in FIG. 5A. It was found that epoxomicin or MG132 alone did not affect NS5A protein levels in 9-13 cells. Epoxomicin (5, 10 μM) and MG132 (10, 20 μM) completely abrogated the degradation of NS5A in cells exposed to a lower concentration of ZnMP (5 μM). In contrast, cells treated with ZnMP (10 μM) and epoxomicin (5, 10 μM) or MG132 (10, 20 μM) displayed significant reversal of the degradation of NS5A by ZnMP, suggesting that the proteasome-dependent degradation pathway is involved in ZnMP-mediated NS5A breakdown. The highest concentration of epoxomicin used in these examples was 10 μM, because cells exposed to 20 μM epoxomicin failed to grow well, indicating that this concentration of epoxomicin was toxic to the cells. In FIG. 5B, The intensities of bands in FIG. 5A were quantified by densitometry, and the relative mean intensities±SE were calculated from three experiments and plotted. The amounts of NS5A protein were normalized to those for GAPDH, which did not vary with treatment. The band intensity of NS5A from vehicle alone was set equal to 1. * differs from vehicle only, P<0.05.

### Example 5

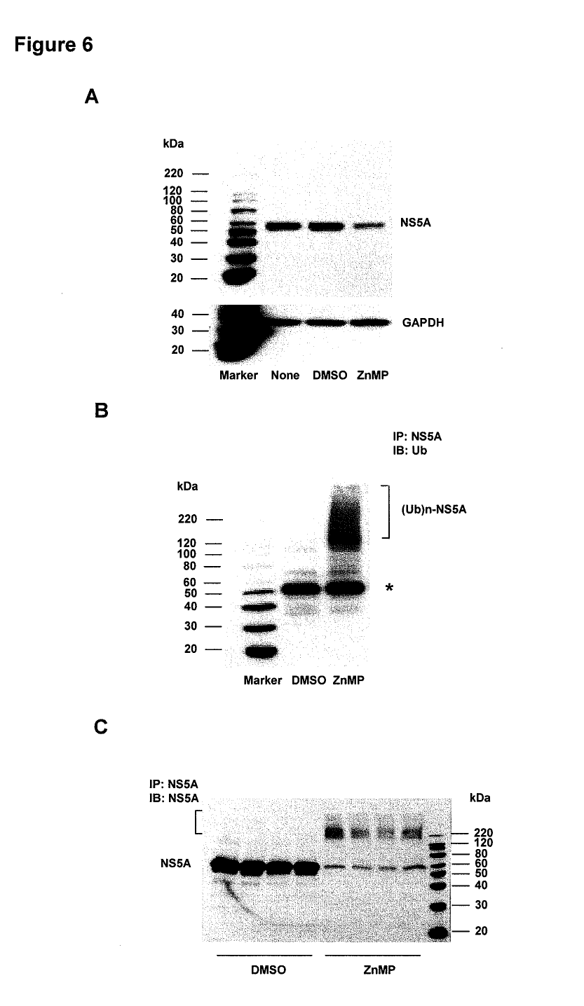

This Example investigates whether ZnMP induces polyubquitination of NS5A to gain insight into the mechanism by which ZnMP mediates degradation of NS5A protein. 9-13 cells were treated without or with 10 μM ZnMP for 4 h. Total proteins were extracted for subsequent Western blot or immunoprecipitation analysis. Immunoprecipitation was carried out using anti-HCV NS5A antibody. Ubiquitin conjugation of NS5A [polyubiquitinated NS5A (Ub)n-NS5A]was examined with immunoprecipitation using an anti-HCV NS5A antibody and immunoblot using an anti-ubiquitin antibody. Western blot analysis of NS5A protein levels before immunoprecipitation shows down-regulation of NS5A by ZnMP (FIG. 6A). In FIG. 6B, ubiquitination of NS5A following ZnMP treatment, or vehicle (DMSO) only was compared. The positions of molecular mass markers (in kilodaltons) are indicated to the left of the gel. The bracket indicates polyubiquitinated NS5A. Asterisks indicate cross-reacting immunoglobulin heavy chain. FIG. 6C shows an immunoblot analysis with an anti-NS5A antibody, indicating that NS5A proteins were immunoprecipitated in panel B. The bracket indicates lower-mobility bands containing NS5A. These bands may represent polyubiquitinated NS5A.

The results suggest that ZnMP induce polyubquitination of NS5A which contributes to the degradation of NS5A by the zinc mesoporphyrin.

### Example 6

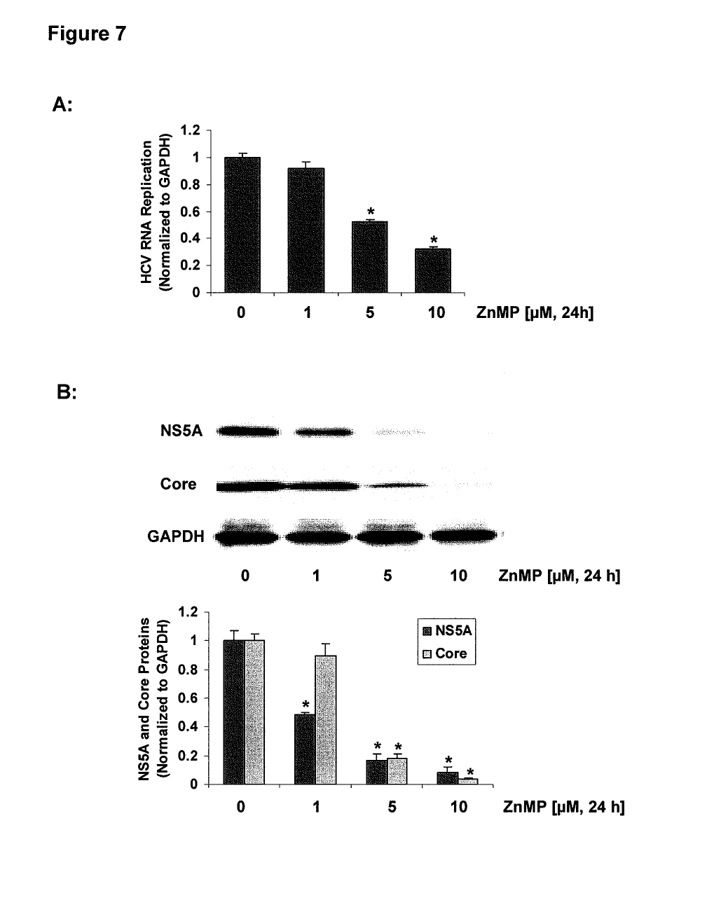

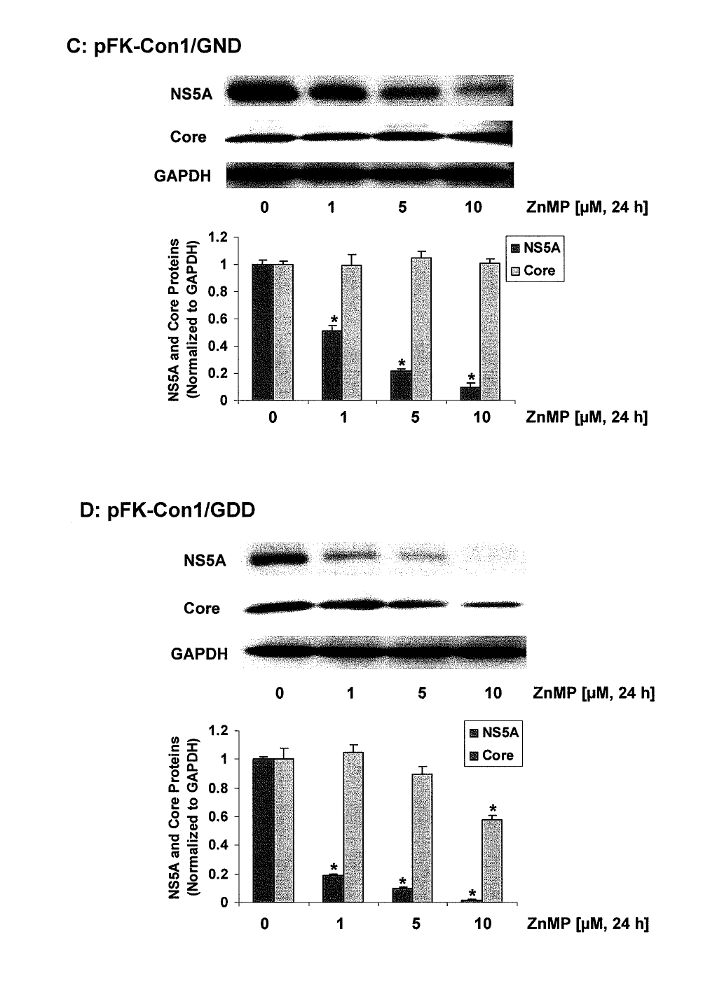

To evaluate whether ZnMP-mediated degradation of NS5A may play a role in inhibiting HCV replication, Con1 full-length HCV replicon Huh-7.5 cells were treated with different concentrations of ZnMP for 24 hours. Total RNA and proteins were extracted. HCV virus RNA was quantified by qRT-PCR, and the levels of HCV core, NS5A and GAPDH protein were measured by Western blots. Data are presented as means±SE, n=3. * differs from vehicle only (ZnMP, 0 μM), P<0.05. The control vehicle alone did not alter the amounts of HCV replicon RNA, whereas treatment with ZnMP resulted in a dose-dependent reduction in viral RNA levels (FIG. 7A), and HCV protein levels (FIG. 7B), suggesting that ZnMP-mediated rapid degradation of NS5A may lead to reduction of HCV RNA replication, and subsequent decrease in HCV protein expression. And then we asked if NS5A is an actual target of ZnMP and the effects of ZnMP on HCV RNA replication and core protein levels are secondary to ZnMP-mediated rapid degradation of NS5A. To this end, we performed parallel experiments with HCV proteins expressed from a DNA plasmid pFK-Con1/GND in Huh-7/T7 cells, where their expression would not be linked to viral RNA polymerase but only to T7 RNA polymerase. ZnMP markedly decreased NS5A protein levels in a dose-dependent fashion, whereas HCV core protein levels remained unaffected after 24 h of ZnMP treatment (FIG. 7C). We further observed that ZnMP resulted in reduction of core in the system that HCV proteins were expressed from pFK-Con1/GDD in Huh-7/T7 cells, where their expression would be partly linked to viral RNA polymerase (FIG. 7D), however, the reduction of core was much less than the effect in Con1 replicon system, where expression of HCV proteins were linked to viral RNA polymerase.

### Example 7

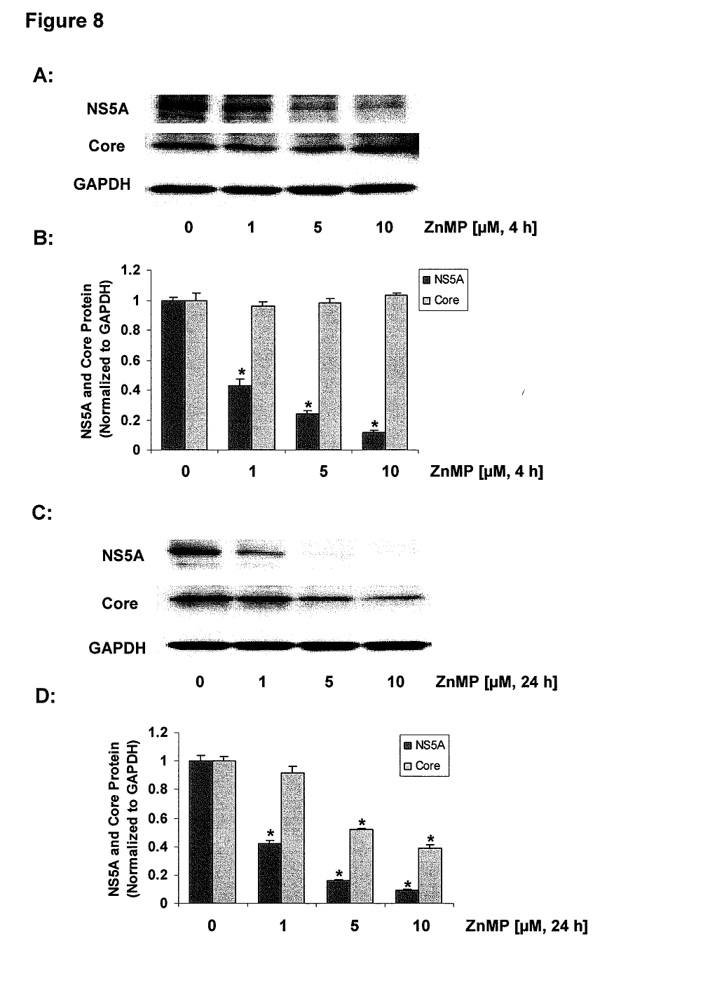

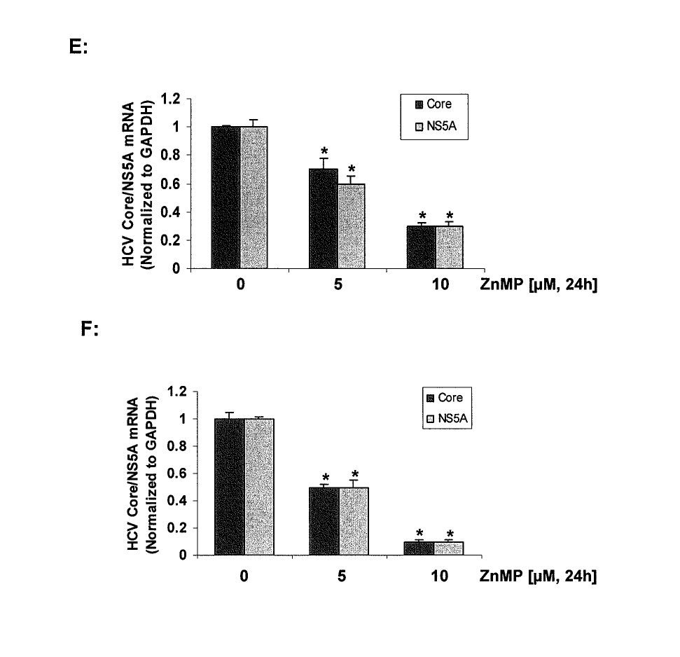

This Example investigates whether ZnMP down-regulates NS5A protein and displays anti-viral activity in the novel JFH1-based (genotype 2a) HCV cell culture system. Huh-7.5 cells were transfected with 2 μg/well of J6/JFH1 RNA by Lipofectamine 2000. After 48 h, cells were treated with indicated concentrations of ZnMP, or DMSO as control for 4 or 24 h, cells were harvested and total RNA and proteins were extracted. HCV RNA was quantified by qRT-PCR, and HCV core, NS5A and GAPDH protein levels were measured by Western blots. ZnMP led to a rapid and profound decrease of NS5A protein levels, while core protein levels were not affected after 4 h of ZnMP treatment and showed a decrease after 24 h exposure to ZnMP (FIGS. 8A-8D). To further examine whether ZnMP inhibits HCV RNA replication/infection in J6/JFH1 transfected and infected cell culture system, we analyzed HCV RNA expression after ZnMP treatment. 10 μM of ZnMP markedly decreased HCV RNA levels by ˜70% in HCV-transfected cells and ˜90% in HCV-infected cells (FIGS. 8E and 8F).

### Example 8

This Example explored the effects of combining ZnMP with Interferon to determine whether there is an additive or synergistic effect of Interferon (IFN) in combination with ZnMP, compared with results for ZnMP or IFN alone. 9-13 cells were treated with 10 μM ZnMP or IFN α, or a combination of both for different times (0, 2, 4, 6, 10, 24 hours), and then the cells were harvested using harvest buffer containing the protease inhibitor cocktail. 50 μg of protein were loaded on a 4-15% SDS-polyacrylamide gel, transferred to a PVDF membrane, and probed with anti-NS5A, and anti-GAPDH specific antibodies, and then developed with ECL Plus reagent. The relative amounts of NS5A protein were normalized to those for GAPDH, which did not vary with treatment. Values for cells treated with vehicle only were set equal to 1. Data are presented as means±SE from triplicate samples. * differs from vehicle only, P<0.05, # differs from IFN alone or ZnMP alone, P<0.05.

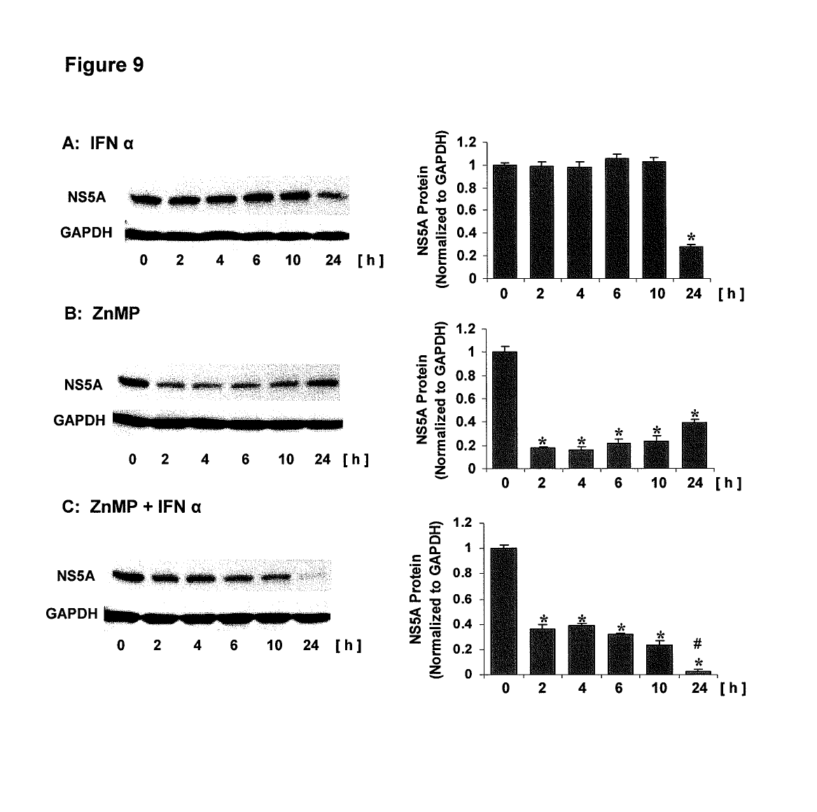

As can be seen in FIG. 9A, the IFN treatment for 24 hours significantly decreased NS5A protein levels, whereas no effects on NS5A protein levels were detectable in cells treated with IFN for less than 10 hours. In FIG. 9B, it can be seen that ZnMP induced rapid and marked down-regulation of NS5A protein levels in cells treated for as little as 2 hours, while NS5A protein was slightly increased after treatment with ZnMP for 10 and 24 hours. As shown in FIG. 9C, A combination of INF and ZnMP revealed an additive effect on NS5A protein expression, compared with results for ZnMP or IFN alone in cells treated for 24 hours. Accordingly, it can be seen that the combination of INF and ZnMP has an additive and/or synergistic effect for the treatment of HCV infection in comparison to a treatment of either alone. Further, it can be seen that this effect is a long duration test extending from as little as 2 hours of treatment to in excess of 24 hours.

### Example 9

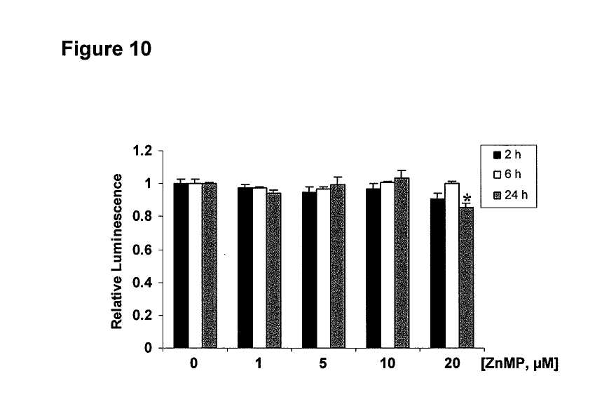

In this Example, the cytotoxicity of metalloporphyrins was evaluated. 9-13 cells were seeded into a 96-well plate 24 hours before treatment. Cells were incubated with the indicated concentrations of ZnMP for 0, 2, 6, 24 hours, and CellTiter-Glo® Reagent was added for CellTiter-Glo luminescent cell viability assay on a Synergy HT microplate reader with integration time set for 0.25 to 1 second. Decreases in luminescence were taken as an index of cellular cytotoxicity. * differs from vehicle, P<0.05, Data represent means±SE of triplicate determinations. As can be seen in FIG. 10, ZnMP (1-10 μM) for 2-24 hours or ZnMP (20 μM) for 2-6 hours had no significant effect on cell viability, whereas ZnMP at a concentration of 20 μM for 24 hours caused significant cytotoxicity in 9-13 cells (p<0.05). Therefore, ZnMP concentrations not exceeding 20 μM for up to 6 hours or up to 10 μM for up to 24 hours were used.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A method of treating a mammal suffering from hepatitis C viral infection comprising reducing NS5A protein levels in cells infected with hepatitis C virus (HCV).

2. The method of claim 1, further comprising the step of enhancing polybubquitination of the NS5A protein.

3. The method of claim 1, further comprising the step of suppressing HCV by treating the infected cells with a metalloporphyrin.

4. The method of claim 3, wherein the metalloporphyrin is selected from the group consisting of zinc mesoporphyrin, zinc protoporphyrin cobalt mesoporphyrin, cobalt mesoporphyrin, and combinations thereof.

5. The method of claim 3, wherein the NS5A protein has a half-life that is reduced to between about 0.5 to 3 hours.

6. The method of claim 3, wherein the NS5A protein has a half-life that is reduced to about 1 to 1.2 hours.

7. A method for the treatment of hepatitis C viral infection in a host in need thereof comprising administration to said host a therapeutically effective amount of a metalloporphyrin.

8. The method of claim 7, wherein the metalloporhyrin is selected from the group consisting of zinc mesoporphyrins, zinc protoporphyrins, heme, zinc deuteroporphyrin, zinc deuteroporphyrin bisglycol, cobalt protoporphyrin, cobalt mesoporphyrin, cobalt deuteroporphyrin, cobalt deuteroporphyrin bisglycol, heme, iron mesoporphyrin, iron deuteroporphyrin, and iron deuteroporphyrin bisglycol.

9. The method of claim 7, wherein an amount of NS5A protein in HCV infected cells is reduced from about 60 to 95%.

10. The method of claim 7, wherein the metallophorpyrin comprises an albumin complex.

11. The method of claim 7, further comprising the step of administering an interferon.

12. The method of claim 7, further comprising the step of reducing a half life of NS5A protein in infected HCV cells to about 1 to 1.2 hours.

13. The method of claim 7, wherein the amount of metalloporphyrin administered is from about 0.1 to 20 milligrams per killogram of the host body weight.

14. The method of claim 7, further comprising the step of administering the metalloporphyrin orally.

16. A method for the treatment of HCV infection in a patient in need thereof comprising administration to said host a therapeutically effective amount of an active ingredient selected from the group consisting of zinc mesoporphyrins, zinc protoporphyrins, heme, zinc deuteroporphyrin, zinc deuteroporphyrin bisglycol, cobalt protoporphyrin, cobalt mesoporphyrin, cobalt deuteroporphyrin, cobalt deuteroporphyrin bisglycol, heme, iron mesoporphyrin, iron deuteroporphyrin, and iron deuteroporphyrin bisglycol.

17. The method of claim 16, further comprising the step of administering an interferon.

18. The method of claim 16, further comprising the step of reducing a half life of NS5A protein in infected HCV cells to about 1 to 1.2 hours.

19. The method of claim 16, wherein the amount of metalloporphyrin administered is from about 0.1 to 20 milligrams per killogram of the patient body weight.

20. A pharmaceutical formulation for the treatment of HCV infection comprising a about 10 to 80 milligrams of zinc protoporphyrin that is bound to human serum albumin in a molar ratio from about 10:1 to 1:1.
